(19) United States
(12) Patent Application Publication
(10) Pub. No.: US 2021/0196148 A1
(43) Pub. Date: Jul. 1, 2021

(54) BREATH SENSOR MEASUREMENT METHODS AND APPARATUS

(71) Applicant: Carrot, Inc., Redwood City, CA (US)

(72) Inventors: Allen JAMESON, Sunnyvale, CA (US); Daniel BALBIERZ, Redwood City, CA (US); Eric TRIDAS, Redwood City, CA (US); Brian HEROLD, Redwood City, CA (US); David S. UTLEY, Redwood City, CA (US)

(73) Assignee: Carrot, Inc., Redwood City, CA (US)

(21) Appl. No.: 17/132,581

(22) Filed: Dec. 23, 2020

Related U.S. Application Data
(60) Provisional application No. 62/955,561, filed on Dec. 31, 2019.

(51) Int. Cl. A61B 5/083; A61B 5/091; A61B 5/087; A61B 5/00

(52) U.S. Cl. CPC A61B 5/0836; A61B 5/091; A61B 2560/0257; A61B 5/4845; A61B 5/087

(57) ABSTRACT

Breath sensor measurement methods and apparatus are described. One variation may generally comprise a sampling unit having a breath sampling port, at least one pressure sensor located within the sampling unit and in communication with the breath sampling port, and a processor in communication with the at least one pressure sensor. The processor may be configured to prompt a user with instructions to exhale a sample breath for a first predetermined period of time into the breath sampling port and to inhale for a second predetermined period of time through the breath sampling port. The processor may be configured to measure a pressure change via the pressure sensor over the first and second predetermined periods of time such that the processor determines a total volume of air corresponding to lung capacity of the user based on the pressure change over the first and second predetermined periods of time.

### CROSS-REFERENCE TO RELATED APPLICATIONS

This application claims the benefit of priority to U.S. Prov. App. 62/955,561 filed Dec. 31, 2019, which is incorporated herein by reference in its entirety.

### FIELD OF THE INVENTION

The present invention relates to apparatus and methods for detecting biological parameters from breath samples. In particular, the present invention relates to apparatus and methods for facilitating the collection of breath samples from users via a breath sampling unit and detecting various biological parameters from the breath samples.

### BACKGROUND OF THE INVENTION

The health problems associated with tobacco smoking are well known. Cigarette smoke contains nicotine as well as many other chemical compounds and additives. Tobacco smoke exposes an individual to carbon monoxide (CO) as well as these other compounds, many of which are carcinogenic and toxic to the smoker and those around the smoker. The presence and level of CO in the exhaled breath of the smoker can provide a marker for identifying the overall smoking behavior of that individual as well as provide a marker for their overall exposure to the other toxic compounds.

In order to sample the exhaled breath, a portable breath sensor which is readily carried by the user and which is unobtrusive is desirable. However, the relatively reduced size of the breath sensor also brings a number of challenges in capturing and accurately measuring samples of the exhaled breath. Factors such as moisture content in the breath as well as breath temperature may affect the accuracy of the sensors used to measure the parameters due to the relatively small size.

In order to sample the exhaled breath, a portable breath sensor which is readily carried by the user and which is unobtrusive is desirable. While this portable breath sensor can measure the exhaled carbon monoxide (eCO) values of users, it may not be immediately intuitive to all users how to use this data since it may not be a widely understood metric.

Electrochemical sensors typically contained within portable breath sensors for detecting carbon monoxide levels from exhaled breath. When sampling the eCO from users, the user is typically prompted to follow breath sampling instructions; however, a number of user compliance issues may occur. For instance, users may exhale into the sampling device too early or too late, the exhaled breath may not be detected, the user may exhale too softly into the sampling unit, or the user may inhale through the sampling unit before the start of the sample.

Accordingly, there remains a need for methods and devices which are able to facilitate the sampling of the user breath in order to optimally detect physiologic parameters from the exhaled breath.

### SUMMARY OF THE INVENTION

Certain biometric data of the user may be obtained by non-invasively detecting and quantifying the smoking behavior for a user based on measuring one or more of the user's biometric data such as their exhaled breath for the determining the level of exhaled carbon monoxide (eCO). Such measurements or data collection can use a portable measuring unit or a fixed measuring unit, either of which communicates with one or more electronic devices for performing the quantification analysis. Alternatively, the analysis can be performed in the portable/fixed unit. However, the user may fail to comply with specified breath sampling protocols for obtaining their breath samples. Non-compliance may result from intentional or unintentional use by the user when providing their breath samples. For instance, users may exhale into the sampling device too early or too late, the exhaled breath may not be detected, the user may exhale too softly into the sampling unit, or the user may inhale through the sampling unit before the start of the sample.

Hence, certain mechanisms and methods may be implemented to ensure that the breath samples are provided in a sufficient manner to reduce any errors or erroneous measurements. Furthermore, the sampling unit may be used not only to detect eCO levels (or other biomarker parameters) of the user, but the same sampling unit may be used to determine various other biologic parameters relating to the user's pulmonary health.

Examples of breath sampling devices and methods for determining and quantifying eCO levels from a user are described in further detail in various patents, e.g., U.S. Pat. Nos. 9,861,126; 10,206,572; 10,306,922; 10,335,032, and U.S. Pat. Pub. 2019/0113501, each of which is incorporated herein by reference in its entirety and for any purpose. Any of the devices described may be utilized with the methods and apparatus described herein.

A portable or personal sampling unit may communicate with either a personal electronic device or a computer. Where the personal electronic device includes, but is not limited to a smartphone, cellular phone, or other personal transmitting device designed or programmed for receiving data from the personal sampling unit. Likewise, the computer is intended to include a personal computer, local server, remote server, etc. Data transmission from the personal sampling unit can occur to both or either the personal electronic device and/or the computer. Furthermore, synchronization between the personal electronic device and the computer is optional. Either the personal electronic device, the computer, and/or the personal sampling unit can transmit data to a remote server for data analysis as described herein. Alternatively, data analysis can occur, fully or partially, via a processor contained in a local device such as the sampling unit (or the computer or personal electronic device). In any case, the personal electronic device and/or computer can provide information to the individual, caretaker, or other individual.

The personal sampling unit receives a sample of exhaled air from the individual via a collection entry or opening. Hardware within the personal sampling unit may include any commercially available electrochemical gas sensor that detects CO gas within the breath sample, commercially available transmission hardware that transmits data (e.g., via Bluetooth®, cellular, or other radio waves to provide transmission of data). The transmitted data and associated measurements and quantification are then displayed on either (or both) a computer display or a personal electronic device. Alternatively, or in combination, any of the information can be selectively displayed on the portable sampling unit.

In another variation of the sampling unit, the device may additionally and/or alternatively incorporate one or more spirometers for monitoring or screening for various conditions as well as one or more pressure transducers or pressure sensors in fluid communication with the sample breath. A spirometer may be incorporated into the unit so that it is in fluid communication with a sample breath passing through the flow path to detect and/or monitor parameters. The one or more pressure sensors and/or spirometer may be wired to a processor within the unit or it may be wireles sly in communication with the personal electronic device or computer. The pressure sensors generally convert pressure exerted by the fluid sample into an electrical signal and may comprise any number of various mechanisms, e.g., piezoresistive, capacitive, electromagnetic, piezoelectric, strain-gauge, optical, etc. The spirometer generally quantifies the volume and flow of the fluid and can be used to assess the user's lung function and can help to identify various pulmonary conditions, e.g., asthma, pulmonary fibrosis, cystic fibrosis, COPD, etc.

Additionally, the flow path may include a flow switch to increase or decrease flow resistance along the flow path. When the subject breaths into the device, they may be instructed to exhale as vigorously as possible and the device may translate measured pressure and volume into a flow rate to calculate, e.g., forced vital capacity (FVC) which is the total volume of air that can be forcibly exhaled after full inhalation, forced expiratory volume, 1 second test (FEV1) which is the volume of air that can be forcibly exhaled over a one second duration after full inhalation. Other parameters which may be calculated by the device may include, e.g., FEV1/FVC ratio (FEV1%) which is the ratio of FEV1 to FVC; FEF/FIF which is the ratio of forced expiratory flow (FEF) to forced inspiratory flow (FIF) for determining the flow rate of air coming out of and into the lungs at various points within a spirometry measurement; and peak expiratory flow (PEF) which is the maximum flow rate experienced during the duration of a spirometry test.

Aside from spirometers and pressure sensors, the sampling unit may also incorporate one or more temperature sensors which converts the detected thermal energy from the flow into a corresponding electrical signal. Such temperature sensors may include, e.g., thermistors and thermocouples. The evaluation of the exhaled breath temperature (EBT) from the exhaled breath sample may be used to detect and monitor various pathological processes in the respiratory system of the user such as the detection of fever, detection of asthma, etc.

Using the pressure sensor, one mechanism for using the user's exhalation and inhalation for determining breath test compliance with a breath sampling protocol may ensure the most accurate result for the user. The user may be instructed to hold their breath for a predetermined period of time, e.g., at least 10 seconds or longer, to allow for the CO levels within their lungs to equilibrate with the levels in their blood. The user may then be instructed to exhale their breath into the sampling unit optionally for a predetermined period of time, e.g., 6 to 12 seconds or longer. Because the user may attempt to “trick” the sampling unit into giving a relatively low reading, appropriate application of the exhalation and inhalation detection may prevent such attempts.

With both the exhalation and inhalation, the pressure sensor within the sampling unit may measure the corresponding pressure within the flow chamber and the timing of the corresponding increase and decrease in pressure may also be measured. Hence, the processor in communication with the pressure sensor may accordingly determine whether the user has complied with the prescribed duration and intensity of the breath measurement (e.g., exhalation or inhalation). When an unexpected increase or decrease in the pressure relative to the ambient pressure is detected, the user can be informed and a corrective suggestion may be provided by the device to the user, for instance, inhaling through the sampling unit when an exhalation is expected or when preparing to hold one's breath. While other methods of breath start detection (e.g., temperature, sound, etc.) are unable to capture all modes of non-compliance with a breath sample protocol, use of the pressure sensor 56 and timing may capture such non-compliance.

Another mechanism for utilizing the pressure sensor within the sampling device is to measure the volume of air entering and leaving the breath sensor and using this metric to estimate and/or track lung capacity as an indicator of the user's health. Because the flow of the user's breath sample through the sampling unit may be considered incompressible at the flow rates generally encountered during exhalation and inhalation, the pressure sensor may be used to measure the resulting pressure in order to determine a relationship between the instantaneous flow rate of air through the unit and the corresponding pressure. By integrating over the duration of the breath, the total volume of air passing through the device may be measured. A relationship between the flow rate and pressure measured by the pressure sensor may then be established for both inhalation and exhalation. By prompting the user to perform a complete exhalation and inhalation (or inhalation and exhalation) through the sampling unit, the total lung volume of the user may be determined.

Yet another mechanism is to provide a pressure sensor placed perpendicularly relative to the flow path to measure spirometric parameters of the user's breath sample. If the minimum cross-sectional area of the flow path is known then the flow rate can be calculated using a dynamic pressure measurement. By recording the flow rate over time, the spirometric parameters can be calculated via the processor. The user may be prompted periodically to perform these measurements to inform them of their pulmonary health.

Yet another mechanism is to combine various flow determination methods described above in combination with detecting for eCO (or any other biomarker). For example, the total volume of air passing through the device may be determined and the relationship between the flow rate and pressure for the inhalation and exhalation may also be determined. Furthermore, the flow rate over time may be recorded to calculate spirometric parameters of the user. While the user exhales their sample breath into the sampling unit, the device may be used to measure the user's eCO by measuring the CO present (or any other biomarker). In this manner, the eCO as well as lung parameters may be determined simultaneously using the same sampling unit.

Yet another mechanism is to determine whether the user is blocking any vent holes (e.g., obstructing flow paths) when exhaling into or inhaling through the sampling unit which could alter the measurements. The device may utilize multiple pressure sensors in various portions of the flow path. The device may generally define a primary CO sensor flow path which leads to the electrochemical sensor for determining eCO and a secondary vent path which allows for a portion of the sample breath to be vented. After the user has been instructed to exhale a sample breath into the sampling unit, a pressure within the CO sensor flow path may be measured and a second pressure within the vent flow path may be measured separately from the CO sensor flow path. If any of the vent openings within either flow path are blocked, an error may be generated in the measurement.

The pressure ratio of the vent flow path to the CO sensor flow path, P(VENT)/P(CO), should remain relatively constant across samples with differing flow rates and if the user were to block any of the vent openings, the ratio may be altered. Hence, the pressure ratio P(VENT)/P(CO) may be calculated via the device processor for comparison between samples obtained. If any sample provides a ratio which differs significantly, this may be an indication that some or all of the vent openings may be blocked and that corrective action may need to be taken by the user.

Yet another mechanism includes providing feedback to the user as encouragement. After the user has exhaled their breath into the sampling unit, any number of measurements may be obtained, as described herein (e.g., flow rate, volume, flow pressure, etc.). This information may be provided as user feedback as encouragement or informational entertainment, etc. to provide the user with a more desirable experience. For example, the total volume of air blown through the device may be recorded and reported back to the user in a fun manner (e.g., “you have blown up 25 twelve inch diameter beach balls!”) Another example may provide an indicator such as an auditory tone or visual indicator may also be provided where the indicator is proportional to, e.g., the pressure of the flow such that the user is encouraged to maintain the indicator constant. Yet another example may provide feedback to the user structured as a game where the user may attempt to produce, e.g., the highest pressure flow possible, which is recorded and compared to previous attempts for determining improvement in lung function.

Yet another mechanism includes determining whether any other health issues may be present. After the user has been instructed to provide a breath sample, certain parameters such as temperature may be measured from the exhaled breath. If a rise in the exhaled breath temperature is observed, this may be indication of a health condition. For instance, the device may determine that the user may have a fever or that the user is experience some asthmatic symptoms.

One variation of an apparatus configured to determine pulmonary parameters by a user may generally comprise a sampling unit having a breath sampling port, at least one pressure sensor located within the sampling unit and in communication with the breath sampling port, at least one gas sensor configured to detect an analyte of interest from a sample breath exhaled by the user into the breath sampling port, wherein the at least one gas sensor is positioned in the sampling unit and in fluid communication with at least a portion of the sample breath, and a processor in communication with the at least one pressure sensor and the at least one gas sensor. The processor may be configured to prompt a user with instructions to exhale a sample breath for a first predetermined period of time into the breath sampling port.

The processor may be further configured to measure a pressure change relative to an ambient pressure via the pressure sensor and correlate this pressure to a flow rate. The processor may be further configured receive a measurement from the at least one gas sensor and correlate this measurement to the analyte of interest from the sample breath. The processor may be further configured to calculate a pulmonologic parameter of the user based on the flow rate.

One method for determining pulmonary parameters of a user may generally comprise prompting the user with instructions to exhale a sample breath into a sampling unit for a first predetermined period of time, measuring a first pressure change of the sample breath over the first predetermined period of time via a pressure sensor in communication with the sample breath, correlating the first pressure change to a flow rate via a processor in communication with the pressure sensor, measuring a biological parameter from the sample breath via at least one gas sensor in fluid communication with at least a portion of the sample breath, correlating a measurement of the biological parameter to an analyte of interest via the processor in communication with the at least one gas sensor, and calculating a pulmonologic parameter of the user via the processor based on the flow rate.

One variation of an apparatus configured to determine sampling compliance by a user may generally comprise a sampling unit having a breath sampling port, at least one pressure sensor located within the sampling unit and in communication with the breath sampling port, and a processor in communication with the at least one pressure sensor. The processor may be configured to prompt a user with instructions to exhale a sample breath for a predetermined period of time into the breath sampling port, to measure a pressure change relative to an ambient pressure via the pressure sensor upon sensing the sample breath, and to measure a timing of the pressure change imparted by the sample breath upon the pressure sensor, and the processor may be further configured to compare the timing of the sample breath against the predetermined period of time and to further compare an intensity of the pressure change against the ambient pressure.

One method for determining sampling compliance by a user may generally comprise prompting the user with instructions to exhale a sample breath into a sampling unit for a predetermined period of time, receiving the sample breath through a breath sampling port defined on the sampling unit, measuring a pressure change relative to an ambient pressure via at least one pressure sensor located within the sampling unit and in communication with the breath sampling port upon sensing the sample breath, measuring a timing of the pressure change imparted by the sample breath upon the at least one pressure sensor via a processor in communication with the at least one pressure sensor, comparing the timing of the sample breath against the predetermined period of time, and comparing an intensity of the pressure change against the ambient pressure.

Another variation of an apparatus configured to determine sampling compliance by a user may generally comprise a sampling unit having a breath sampling port, a first pressure sensor located in communication with a primary flow path within the sampling unit and in fluid communication with the breath sampling port, a second pressure sensor located in communication with a secondary flow path within the sampling unit and in fluid communication with the breath sampling port and one or more vent openings, and a processor in communication with the first and second pressure sensors, wherein the processor is configured to obtain a first pressure measurement from the first pressure sensor and a second pressure measurement from the second pressure sensor and determine a pressure ratio of the second pressure measurement to the first pressure measurement.

Another method for determining sampling compliance by a user may generally comprise receiving a sample breath through a sampling port defined on a sampling unit such that a first portion of the sample breath flows into a primary flow path and a second portion of the sample breath flows into a secondary flow path and through one or more vent openings, obtaining a first pressure measurement via a first pressure sensor in the primary flow path, obtaining a second pressure measurement via a second pressure sensor in the secondary flow path, determining a pressure ratio of the second pressure measurement to the first pressure measurement via a processor in communication with the first and second pressure sensors, and comparing the pressure ratio against subsequent pressure ratios obtained from subsequent breath sample measurements for deviations.

### BRIEF DESCRIPTION OF THE DRAWINGS

FIG. 1A illustrates a variation of a system which is able to receive the exhaled breath from a subject and detect various parameters and which can communicate with a one or more remote devices.

FIG. 1B illustrates one variation of the internal circuitry and sensors contained within the housing of the breath sensor.

FIG. 1C illustrates a top view of the flow path control device positioned over the sensors.

FIG. 2 illustrates a detail view of a flow path within the sampling unit and various sensors which may be incorporated.

FIGS. 3A and 3B illustrate graphs showing typical flow parameters during exhalation and inhalation.

FIG. 4 illustrates a flow diagram of one mechanism for determining breath test compliance.

FIG. 5A illustrates a flow diagram of another mechanism for estimating and/or tracking lung capacity as an indicator of the user's health.

FIG. 5B illustrates a flow diagram of another mechanism for estimating and/or tracking pulmonologic parameters and analytes of interest as an indicator of the user's health.

FIG. 6 illustrates a flow diagram of another mechanism for measuring spirometric parameters of the user's breath sample.

FIG. 7 illustrates a flow diagram illustrating yet another method which combines various flow parameter measurements with biomarker detection (e.g., eCO) from the user's breath sample.

FIG. 8 illustrates a flow diagram illustrating yet another method for determining whether the user is blocking any vent holes when exhaling into or inhaling through the sampling unit which could alter the measurements.

FIG. 9 illustrates a flow diagram illustrating a method for providing feedback to the user as encouragement.

FIG. 10 illustrates a flow diagram for methods of determining whether any other health issues may be present.

### DETAILED DESCRIPTION OF THE INVENTION

Certain biometric data of the user may be obtained by non-invasively detecting and quantifying the smoking behavior for a user based on measuring one or more of the user's biometric data such as their exhaled breath for the determining the level of exhaled carbon monoxide (eCO). Such measurements or data collection can use a portable measuring unit or a fixed measuring unit, either of which communicates with one or more electronic devices for performing the quantification analysis. Alternatively, the analysis can be performed in the portable/fixed unit. However, the user may fail to comply with specified breath sampling protocols for obtaining their breath samples. Non-compliance may result from intentional or unintentional use by the user when providing their breath samples. For instance, users may exhale into the sampling device too early or too late, the exhaled breath may not be detected, the user may exhale too softly into the sampling unit, or the user may inhale through the sampling unit before the start of the sample. Hence, certain mechanisms and methods may be implemented to ensure that the breath samples are provided in a sufficient manner to reduce any errors or erroneous measurements. Furthermore, the sampling unit may be used not only to detect eCO levels (or other biomarker parameters) of the user, but the same sampling unit may be used to determine various other biologic parameters relating to the user's pulmonary health.

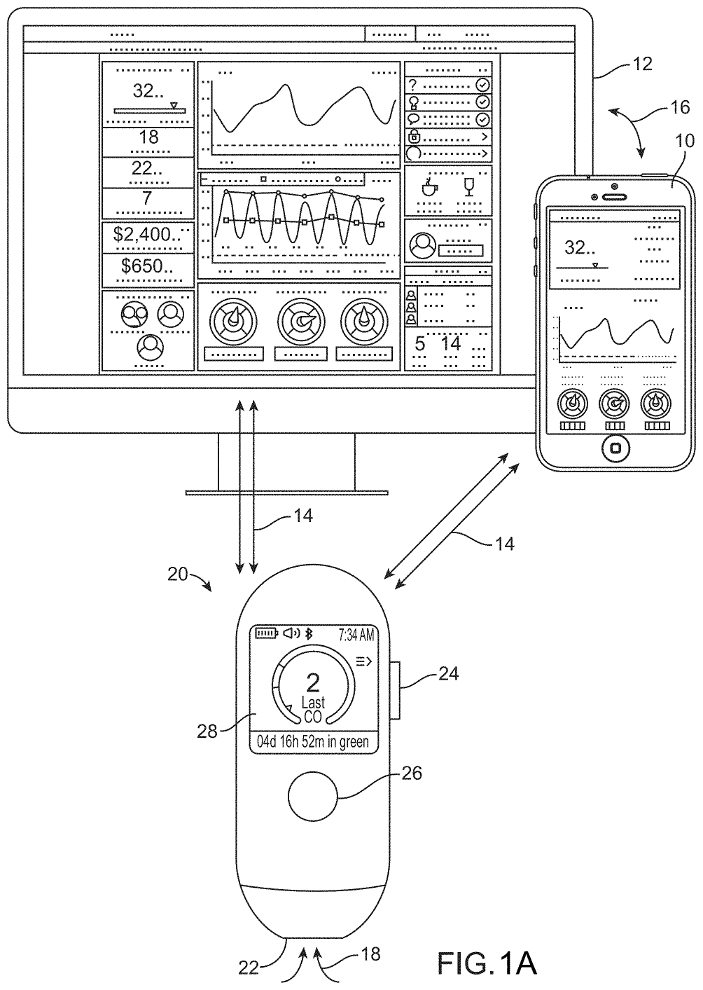

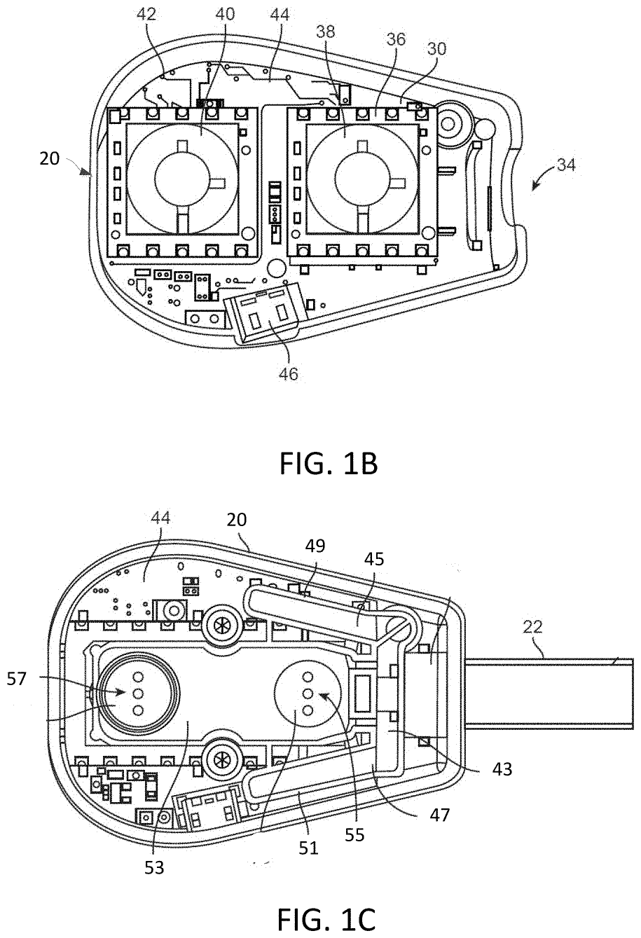

FIG. 1A illustrates one variation of a system and/or method in which a plurality of samples of biometric data are obtained from the user and analyzed to quantify the user's exposure to cigarette smoke such that the quantified information can be relayed to the individual, a medical caregiver, and/or other parties having a stake in the individual's health. The example discussed below employs a portable device 20 that obtains a plurality of samples of exhaled air from the individual with commonly available sensors that measure an amount of eCO within the sample of exhaled air. However, the quantification and information transfer are not limited to exposure of smoking based on exhaled air. As noted above, there are many sampling mechanisms to obtain a user's smoking exposure. The methods and devices described in the present example can be combined or supplemented with any number of different sampling mechanisms where possible while still remaining within the scope of the invention.

The measurement of eCO level has been known to serve as an immediate, non-invasive method of assessing a smoking status of an individual. The eCO levels for non-smokers can range between, e.g., 0 ppm to 6 ppm, or more particularly between, e.g., 3.61 ppm and 5.6 ppm.

As shown, a portable or personal sampling unit 20 may communicate with either a personal electronic device 10 or a computer 12. Where the personal electronic device 10 includes, but is not limited to a smartphone, cellular phone, or other personal transmitting device designed or programmed for receiving data from the personal sampling unit 20. Likewise, the computer 12 is intended to include a personal computer, local server, remote server, etc. Data transmission 14 from the personal sampling unit 20 can occur to both or either the personal electronic device 10 and/or the computer 12. Furthermore, synchronization 16 between the personal electronic device 10 and the computer 12 is optional. Either the personal electronic device 10, the computer 12, and/or the personal sampling unit 20 can transmit data to a remote server for data analysis as described herein. Alternatively, data analysis can occur, fully or partially, via a processor contained in a local device such as the sampling unit 20 (or the computer 12 or personal electronic device 10). In any case, the personal electronic device 10 and/or computer 12 can provide information to the individual, caretaker, or other individual as shown in FIG. 1A.

The personal sampling unit 20 receives a sample of exhaled air 18 from the individual via a collection entry or opening 22. Hardware within the personal sampling unit 20 may include any commercially available electrochemical gas sensor that detects CO gas within the breath sample, commercially available transmission hardware that transmits data 14 (e.g., via Bluetooth®, cellular, or other radio waves to provide transmission of data). The transmitted data and associated measurements and quantification are then displayed on either (or both) a computer display 12 or a personal electronic device 10. Alternatively, or in combination, any of the information can be selectively displayed on the portable sampling unit 20.

The personal sampling unit 20 (or personal breathing unit) can also employ standard ports to allow direct-wired communication with the respective devices 10 and 12. In certain variations, the personal sampling unit 20 can also include memory storage, either detachable or built-in, such that the memory permits recording of data and separate transmission of data. Alternatively, the personal sampling unit can allow simultaneous storage and transmission of data. Additional variations of the device 20 do not require memory storage. In addition, the unit 20 can employ any number of GPS components, inertial sensors (to track movement), and/or other sensors that provide additional information regarding the patient's behavior.

The personal sampling unit 20 can also include any number of input trigger (such as a switch or sensors) 24, 26. As described below, the input trigger 24, 26 may allow the individual to prime the device 20 for delivery of a breath sample 18 or to record other information regarding the cigarette such as quantity of cigarette smoked, the intensity, etc. In addition, variations of the personal sampling unit 20 may also associate a timestamp of any inputs to the device 20. For example, the personal sampling unit 20 can associate the time at which the sample is provided and provide the measured or inputted data along with the time of the measurement when transmitting data 14. Alternatively, the personal sampling device 20 can use alternate mechanisms to identify the time that the sample is obtained. For example, given a series of samples rather than recording a timestamp for each sample, the time periods between each of the samples in the series can be recorded. Therefore, identification of a timestamp of any one sample allows determination of the time stamp for each of the samples in the series.

In certain variations, the personal sampling unit 20 may be designed such that it has a minimal profile and can be easily carried by the individual with minimal effort. Therefore the input triggers 24 can comprise low profile tactile switches, optical switches, capacitive touch switches, or any commonly used switch or sensor. The portable sampling unit 20 can also provide feedback or information to the user using any number of commonly known techniques. For example, as shown, the portable sampling unit 20 can include a screen 28 that shows select information as discussed below. Alternatively or additionally, the feedback can be in the form of a vibrational element, an audible element, and a visual element (e.g., an illumination source of one or more colors). Any of the feedback components can be configured to provide an alarm to the individual, which can serve as a reminder to provide a sample and/or to provide feedback related to the measurement of smoking behavior. In addition, the feedback components can provide an alert to the individual on a repeating basis in an effort to remind the individual to provide periodic samples of exhaled air to extend the period of time for which the system captures biometric (such as eCO, CO levels, H₂ etc.) and other behavioral data (such as location either entered manually or via a GPS component coupled to the unit, number of cigarettes, or other triggers). In certain cases, the reminders can be triggered at higher frequency during the initial program or data capture. Once sufficient data is obtained, the reminder frequency can be reduced.

In obtaining the breath sample with the sampling unit 20, instructions may be provided on the personal electronic device 10 or computer display 12 for display to the subject in a guided breath test for training the subject to use the unit 20. Generally, the subject may be instructed, e.g., on the screen 28 of the electronic device 10, to first inhale away from the unit 20 and then to exhale into the unit 20 for a set period of time. The unit 20 may optionally incorporate one or more pressure sensors fluidly coupled with, e.g., check valves, to detect if the subject inhales through the unit 20.

FIG. 1B shows the sampling unit 20 with a portion of the housing 30 and collection entry or opening 22 removed to show a top view of the electrochemical sensors contained within. In this variation, a first sensor 38 and second sensor 42 (either or both of the sensors 38, 42 may include CO and H₂ sensors) are shown optionally positioned upon respect respective sensor platforms 36, 40 which in turn may be mounted upon a substrate such as a printed circuit board 44. Although in other variations, one or more sensors may be used depending upon the parameters being detected. In other variations, the one or more sensors may be mounted directly upon the printed circuit board 44. A power port and/or data access port 46 may also be seen integrated with the printed circuit board 44 and readily accessible by a remote device such as a computer, server, smartphone, or other device. As shown, multiple sensors 38, 42 or a single sensor may be used to detect the parameters from the sampled breath.

In other variations, at least one CO sensor or multiple CO sensors may be implemented alone. Alternatively, one or more CO sensors may be used along with one or more H₂ sensors in combination. If both a CO and H₂ sensor are used, the readings from the H₂ sensor may be used to account for or compensate for any H₂ signals detected by the CO sensor since many CO sensors have a cross-sensitivity to H₂ which is frequently present in sufficient quantity to potentially affect CO measurement in the breath of people. If a CO sensor is used without an H₂ sensor, various methods may be applied to reduce any H₂ measurement interference to a nominally acceptable level. However, the use of an H₂ sensor to directly measure and compensate for the presence of H₂ may facilitate CO measurement. The sensors may also include any number of different sensor types including chemical gas sensors, electrochemical gas sensors, etc. for detecting agents such as carbon monoxide in the case of detecting smoking related inhalation.

FIG. 1C shows a top view of the flow control assembly incorporated into the housing 20 and sealed into positioned over the sensors such that the sampled air entering through the lumen is contained within the sampling unit. The sample breath may enter the device when exhaled by the subject. The breath enters a dispersion chamber 43 where a majority of the sample, e.g., about 80%, is diverted through the chamber 43 and into respective secondary fluid pathways 45, 47 defined by secondary channels 49, 51. The remaining sample, about 20%, enters through the primary channels and into the receiving channel 53 where the breath may then enter through the openings 55, 57 and into contact with the sensors. In other variations, more than 50% of the breath sample may be diverted so that less than 50% of the breath sample enters into the receiving channel.

Further examples of breath sampling devices and methods for determining and quantifying eCO levels from a user are described in further detail in various patents, e.g., U.S. Pat. Nos. 9,861,126; 10,206,572; 10,306,922; 10,335,032, and U.S. Pat. Pub. 2019/0113501, each of which is incorporated herein by reference in its entirety and for any purpose. Any of the devices described may be utilized with the methods and apparatus described herein.

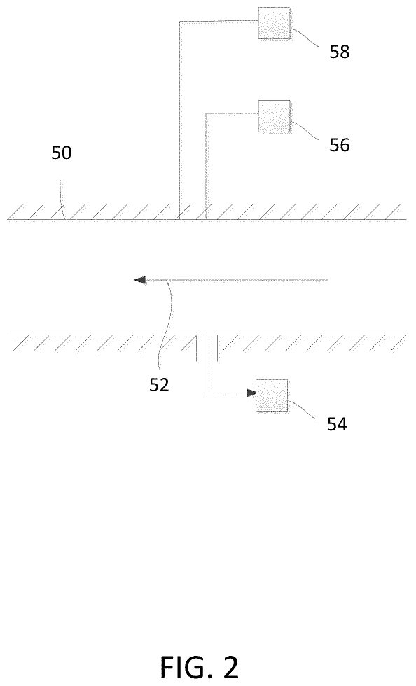

In another variation of the sampling unit 20, the device may additionally and/or alternatively incorporate one or more spirometers 54 for monitoring or screening for various conditions as well as one or more pressure transducers or pressure sensors 56 in fluid communication with the sample breath 52. Although a single pressure sensor 56 is illustrated, additional pressure sensors may be incorporated at various positions within the sampling unit 20. The spirometer 54 may be incorporated into the unit 20 so that it is in fluid communication with a sample breath 52 passing through the flow path 50 to detect and/or monitor parameters, as shown in FIG. 2. The one or more pressure sensors 56 and/or spirometer 54 may be wired to a processor within the unit 20 or it may be wirelessly in communication with the personal electronic device 10 or computer 12. The pressure sensors 56 generally convert pressure exerted by the fluid sample 52 into an electrical signal and may comprise any number of various mechanisms, e.g., piezoresistive, capacitive, electromagnetic, piezoelectric, strain-gauge, optical, etc. The spirometer 54 generally quantifies the volume and flow of the fluid 52 and can be used to assess the user's lung function and can help to identify various pulmonary conditions, e.g., asthma, pulmonary fibrosis, cystic fibrosis, COPD, etc.

Additionally, the flow path 50 may include a flow switch to increase or decrease flow resistance along the flow path. When the subject breaths into the device, they may be instructed to exhale as vigorously as possible and the device may translate measured pressure and volume into a flow rate to calculate, e.g., forced vital capacity (FVC) which is the total volume of air that can be forcibly exhaled after full inhalation, forced expiratory volume, 1 second test (FEV1) which is the volume of air that can be forcibly exhaled over a one second duration after full inhalation. Other parameters which may be calculated by the device may include, e.g., FEV1/FVC ratio (FEV1%) which is the ratio of FEV1 to FVC; FEF/FIF which is the ratio of forced expiratory flow (FEF) to forced inspiratory flow (FIF) for determining the flow rate of air coming out of and into the lungs at various points within a spirometry measurement; and peak expiratory flow (PEF) which is the maximum flow rate experienced during the duration of a spirometry test.

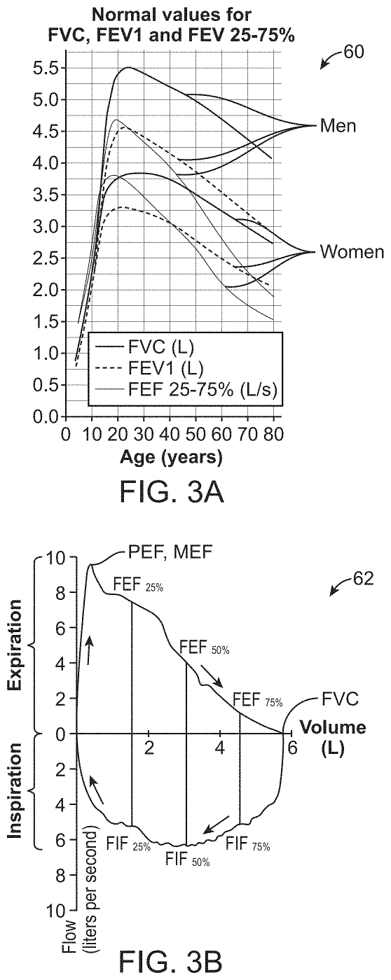

FIG. 3A shows a graph 60 illustrating normal values for FVC, FEV1 and FEV 25-75% over various ages for both men and women and FIG. 3B shows graph 62 illustrating the typical flow (liters per second) over volume (L) for FEF at 25%, 50%, and 75% during expiration and FIF at 75%, 50%, and 25% during inspiration for reference.

Aside from spirometers 54 and pressure sensors 56, the sampling unit may also incorporate one or more temperature sensors 58, as shown in FIG. 2, which converts the detected thermal energy from the flow 52 into a corresponding electrical signal. Such temperature sensors 58 may include, e.g., thermistors and thermocouples. The evaluation of the exhaled breath temperature (EBT) from the exhaled breath sample may be used to detect and monitor various pathological processes in the respiratory system of the user such as the detection of fever, detection of asthma, etc.

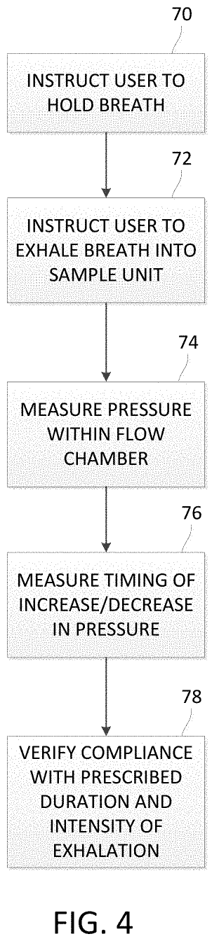

Using the pressure sensor 56, one mechanism for using the user's exhalation and inhalation for determining breath test compliance is shown in the flow diagram of FIG. 4 as user compliance with a breath sampling protocol may ensure the most accurate result for the user. As shown, the user may be instructed to hold their breath 70 for a predetermined period of time, e.g., at least 10 seconds or longer, to allow for the CO levels within their lungs to equilibrate with the levels in their blood. The user may then be instructed to exhale their breath into the sampling unit 72 optionally for a predetermined period of time, e.g., 6 to 12 seconds or longer. Because the user may attempt to “trick” the sampling unit into giving a relatively low reading, appropriate application of the exhalation and inhalation detection may prevent such attempts.

With both the exhalation and inhalation, the pressure sensor 56 within the sampling unit may measure the corresponding pressure within the flow chamber 74 and the timing of the corresponding increase and decrease in pressure may also be measured 76. Hence, the processor in communication with the pressure sensor 56 may accordingly determine whether the user has complied with the prescribed (or expected) duration and intensity of the breath measurement (e.g., exhalation or inhalation) 78 by comparing relative to the duration and intensity of the actual measured sample breath. For instance, the actual timing of the sample breath against the prescribed predetermined period of time for exhaling into the device may be compared for timing and an intensity of the measured pressure change against an ambient pressure level may be compared for breath intensity. When an unexpected increase or decrease in the pressure relative to the ambient pressure is detected, and/or when the timing of the sample breath falls outside the predetermined period of time, the user can be informed and a corrective suggestion may be provided by the device to the user, for instance, inhaling through the sampling unit 20 when an exhalation is expected or when preparing to hold one's breath. While other methods of breath start detection (e.g., temperature, sound, etc.) are unable to capture all modes of non-compliance with a breath sample protocol, use of the pressure sensor 56 and timing may capture such non-compliance.

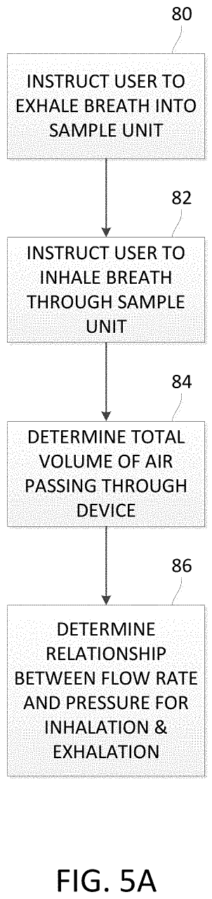

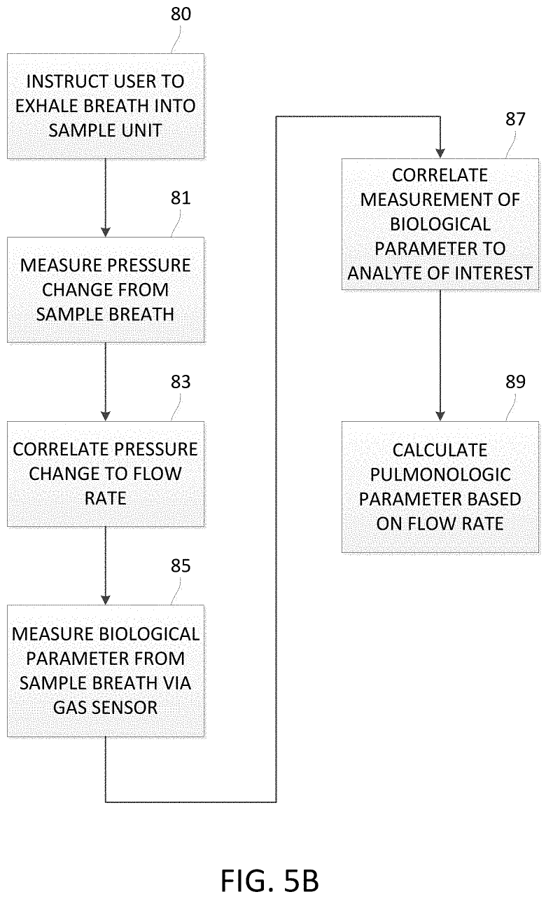

FIG. 5A shows a flow diagram of another mechanism for utilizing the pressure sensor 56 within the sampling device 20 to measure the volume of air entering and leaving the breath sensor and using this metric to estimate and/or track lung capacity as an indicator of the user's health. Because the flow of the user's breath sample through the sampling unit 20 may be considered incompressible at the flow rates generally encountered during exhalation and inhalation, the pressure sensor 56 may be used to measure the resulting pressure in order to determine a relationship between the instantaneous flow rate of air through the unit 20 and the corresponding pressure. The user may be instructed to exhale a breath 80 into the sampling unit and further inhale a breath 82 through the sampling unit 20. Optionally, the user may be further instructed to exhale through the unit 20 once again so that a full exhale-inhale-exhale cycle through the unit 20 may be obtained to measure lung volume parameters. By integrating over the duration of the breath, the total volume of air passing through the device may be measured 84. A relationship between the flow rate and pressure measured by the pressure sensor 56 may then be established for both inhalation and exhalation 86.

By prompting the user to perform a complete exhalation and inhalation and optionally followed by another exhalation (or inhalation, exhalation, and optionally inhalation again) through the sampling unit 20, the total lung volume of the user may be determined. This process may be performed periodically and monitored to provide the user with feedback on how their lung capacity may be changing over time. Integration to calculate the cumulative exhaled volume over the course of the breath can also aid in making the algorithm more accurate, e.g., estimation of when dead volume is expended and alveolar air is being sampled.

FIG. 5B shows yet another flow diagram of a mechanism for utilizing the pressure sensor 56 within the sampling device 20 and at least one sensor to measure a biologic parameter for determining pulmonologic parameters of the user. As described, the user may be prompted to exhale their breath 80 into the sampling unit 20 for some predetermined period of time, e.g., 6 to 12 seconds or longer. Prior to exhaling, the user may be optionally instructed to hold their breath for a predetermined period of time, e.g., at least 10 seconds or longer, to allow for the CO levels within their lungs to equilibrate with the levels in their blood. As the user exhales their sample breath, the change in pressure imparted by the sample breath may be measured 81 via at least one pressure sensor in communication with the processor within the sampling unit 20 relative to an ambient pressure. The timing of the exhalation may also be measured via the processor. The pressure change may then be correlated to a flow rate 83 via the processor.

One or more biological parameters from the sample breath may also be measured via one or more sensors 85, e.g., gas sensor, which is in fluid communication with at least a portion of the sample breath which is diverted to the one or more sensors contained within the sampling unit 20 (as previously described). This measurement may be obtained simultaneously to the measurement of the flow parameters or the measurement may be obtained from a separate sample breath taken closely in time. The remainder of the sample breath may be optionally vented from the unit 20. The measurements of the biological parameters obtained from the one or more sensors may be correlated to an analyte of interest 87 via the processor where the analyte may comprise CO level from the sample breath or any number of other analytes, e.g., H₂, CH₄, CO₂, O₂, C₃H₆O, etc. which are indicative of a corresponding biological parameter of the user. The pulmonologic parameter of the user may then be calculated via the processor based on the flow rate 89 obtained from the correlated pressure change. Hence, the pulmonologic parameter and the corresponding biological parameter may be obtained from a single sample breath. Subsequent pulmonologic and biological parameters may be obtained from the user over a predetermined period of time for the purposes of tracking the user's lung health which may be provided as feedback to the user.

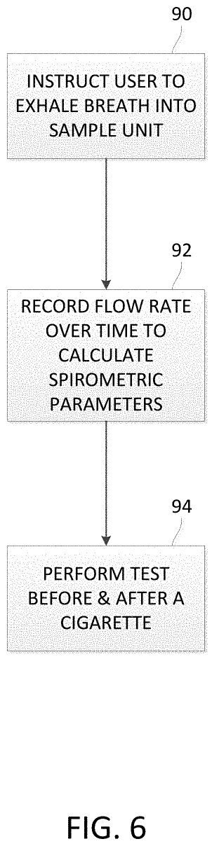

FIG. 6 shows another flow diagram illustrating how a pressure sensor 56 (e.g., placed perpendicularly relative to the flow path) may be used to measure spirometric parameters of the user's breath sample. The user may be instructed to exhale through the sampling unit 90 to provide a sample breath. Typically, flow parameters may be difficult to assess in devices having a relatively high flow resistance, but orienting the pressure sensor 56 perpendicularly relative to the direction of flow may enable the device to calculate the flow velocity using the following dynamic pressure equation:

$$q = \frac{ρ {u}^{2}}{2} \qquad ( 1 )$$

where,
q=dynamic pressure (Pa)
ρ=fluid density of air
u=velocity of flow
   Rearranging the equation (1) to solve for the velocity of flow yields the following equation:

$$u = \sqrt{\frac{2 q}{ρ}} \qquad ( 2 )$$

If the minimum cross-sectional area of the flow path is known then the flow rate can be calculated using the dynamic pressure measurement using equation (2). By recording the flow rate over time, the spirometric parameters can be calculated 92 via the processor.

The user may be prompted periodically to perform these measurements to inform them of their pulmonary health. For instance, the user may be prompted to perform these tests before and after smoking a cigarette 94 to provide feedback on the immediate effects that cigarette smoking has on their health. Recording these values over time can also help encourage users to continue reducing their cigarette intake as their spirometry measurements improve.

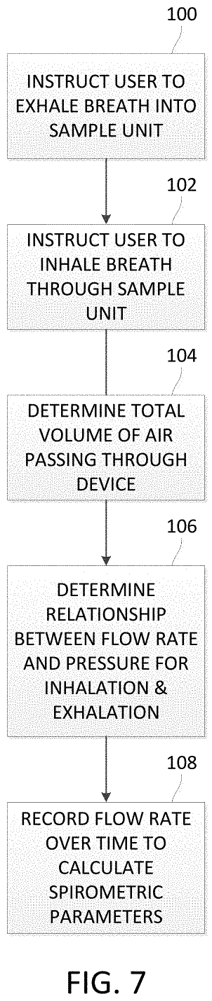

FIG. 7 shows another flow diagram illustrating yet another method which combines those described above with respect to FIGS. 5 and 6. As above, the user may be instructed to exhale a sample breath into the sampling unit 100 and to further inhale a breath through the sampling unit 102. As described, the total volume of air passing through the device may be determined 104 and the relationship between the flow rate and pressure for the inhalation and exhalation may also be determined 106. Furthermore, the flow rate over time may be recorded to calculate spirometric parameters 108 of the user. While the user exhales their sample breath into the sampling unit 100, the device may be used to measure the user's eCO by measuring the CO present (or any other biomarker). In this manner, the eCO as well as lung parameters may be determined simultaneously using the same sampling unit 20.

Each method may utilize differing flow resistances which may utilize distinct flow path geometries through the sampling unit 20 and this may dictate the biomarker sampling method employed. As above, the device may calculate expiratory and inspiratory lung volumes and also calculate various spirometry measurements.

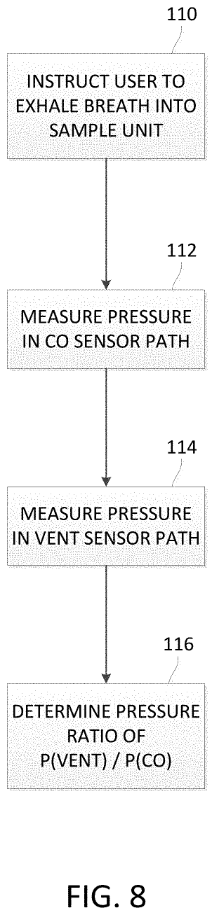

FIG. 8 shows yet another flow diagram illustrating methods which may be utilized to determine whether the user is blocking any vent holes (e.g., obstructing flow paths) when exhaling into or inhaling through the sampling unit 20 which could alter the measurements. The device may utilize multiple pressure sensors in various portions of the flow path. The device may generally define a primary CO sensor flow path which leads to the electrochemical sensor for determining eCO and a secondary vent path which allows for a portion of the sample breath to be vented, as shown and described herein with respect to FIG. 1C. After the user has been instructed to exhale a sample breath into the sampling unit 110, a pressure within the CO sensor flow path may be measured 112 and a second pressure within the vent flow path may be measured separately from the CO sensor flow path 114. If any of the vent openings within either flow path are blocked, an error may be generated in the measurement.

The pressure ratio of the vent flow path to the CO sensor flow path, P(VENT)/P(CO), should remain relatively constant across samples with differing flow rates and if the user were to block any of the vent openings, the ratio may be altered. Hence, the pressure ratio P(VENT)/P(CO) may be calculated 116 via the device processor for comparison between samples obtained. If any sample provides a ratio which differs significantly, this may be an indication that some or all of the vent openings may be blocked and that corrective action may need to be taken by the user.

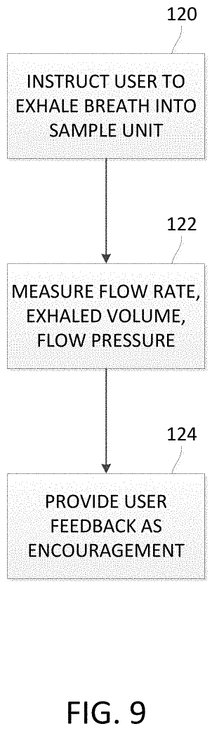

FIG. 9 shows yet another flow diagram which illustrates a method for providing feedback to the user as encouragement. After the user has exhaled their breath into the sampling unit 120, any number of measurements may be obtained, as described herein (e.g., flow rate, volume, flow pressure, etc.) 122. This information may be provided as user feedback 124 as encouragement or informational entertainment, etc. to provide the user with a more desirable experience. For example, the total volume of air blown through the device may be recorded and reported back to the user in a fun manner (e.g., “you have blown up 25 twelve inch diameter beach balls!”) Another example may provide an indicator such as an auditory tone or visual indicator may also be provided where the indicator is proportional to, e.g., the pressure of the flow such that the user is encouraged to maintain the indicator constant. Yet another example may provide feedback to the user structured as a game where the user may attempt to produce, e.g., the highest pressure flow possible, which is recorded and compared to previous attempts for determining improvement in lung function.

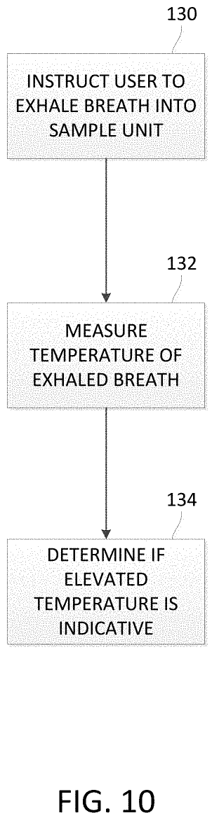

FIG. 10 shows yet another flow diagram for methods of determining whether any other health issues may be present. After the user has been instructed to provide a breath sample 130, certain parameters such as temperature may be measured from the exhaled breath 132. If a rise in the exhaled breath temperature is observed, this may be indication of a health condition 134. For instance, the device may determine that the user may have a fever or that the user is experience some asthmatic symptoms.

Any of the methods and mechanisms described, for instance, with respect to FIGS. 4 to 10 may be combined with any of the physiologic measurement devices (e.g., pressure, spirometry, temperature, etc.) in any number of combinations to effect combinational assessments, measurements, etc. and are intended to be within the scope of this description. For instance, the physiologic measurement devices may also incorporate any number of additional sensors for detecting various other parameters such as CO (as described), H₂, CH₄ (methane), CO₂, O₂, C₃H₆O (acetone), etc. as indicative of various biological functions.

While illustrative examples are described above, it will be apparent to one skilled in the art that various changes and modifications may be made therein. Moreover, various apparatus or procedures described above are also intended to be utilized in combination with one another, as practicable. The appended claims are intended to cover all such changes and modifications that fall within the true spirit and scope of the invention.

What is claimed is:

1. An apparatus configured to measure pulmonary parameters of a user, comprising:
a sampling unit having a breath sampling port;
at least one pressure sensor located within the sampling unit and in communication with the breath sampling port;
at least one gas sensor configured to detect an analyte of interest from a sample breath exhaled by the user into the breath sampling port, wherein the at least one gas sensor is positioned in the sampling unit and in fluid communication with at least a portion of the sample breath,
a processor in communication with the at least one pressure sensor and the at least one gas sensor,
wherein the processor is configured to prompt a user with instructions to exhale a sample breath for a first predetermined period of time into the breath sampling port,
wherein the processor is further configured to measure a pressure change relative to an ambient pressure via the pressure sensor and correlate this pressure to a flow rate,
wherein the processor is further configured to receive a measurement from the at least one gas sensor and correlate this measurement to the analyte of interest from the sample breath, and
wherein the processor is further configured to calculate a pulmonologic parameter of the user based on the flow rate.

2. The apparatus of claim 1 wherein the processor is configured to prompt the user with instructions to exhale at a constant flow rate or constant pressure.

3. The apparatus of claim 1 wherein the processor is configured to further prompt the user with instructions to inhale for a second predetermined period of time through the breath sampling port.

4. The apparatus of claim 1 wherein the processor is further configured to prompt the user with instructions to hold their breath for at least 10 seconds prior to exhaling the sample breath.

5. The apparatus of claim 1 wherein the processor is further configured to prompt a user with instructions to exhale the sample breath for at least 6 to 12 seconds.

6. The apparatus of claim 3 wherein the processor is further configured to prompt the user to exhale for a third predetermined period of time into the breath sampling port.

7. The apparatus of claim 1 wherein the at least one gas sensor is configured to sense a CO level from the sample breath.

8. The apparatus of claim 7 wherein the at least one gas sensor is configured to sense a level of H₂, CH₄, CO₂, O₂, or C₃H₆O.

9. A method of measuring pulmonary parameters of a user, comprising:
prompting the user with instructions to exhale a sample breath into a sampling unit for a first predetermined period of time;
measuring a first pressure change of the sample breath over the first predetermined period of time via a pressure sensor in communication with the sample breath;
correlating the first pressure change to a flow rate via a processor in communication with the pressure sensor;
measuring a biological parameter from the sample breath via at least one gas sensor in fluid communication with at least a portion of the sample breath;
correlating a measurement of the biological parameter to an analyte of interest via the processor in communication with the at least one gas sensor; and
calculating a pulmonologic parameter of the user via the processor based on the flow rate.

10. The method of claim 9 further comprising:
prompting the user with instructions to inhale through the sampling unit for a second predetermined period of time via the pressure sensor;
measuring a second pressure change of the sample breath over the second predetermined period of time; and
determining a total volume of air corresponding to a lung capacity of the user via the processor based on the first and second pressure changes over the first and second predetermined periods of time.

11. The method of claim 9 further comprising comparing the pulmonologic parameter against a subsequent pulmonologic parameter to estimate lung capacity of the user over time.

12. The method of claim 9 further comprising prompting the user with instructions to hold their breath for at least 10 seconds prior to exhaling the sample breath.

13. The method of claim 9 further comprising prompting the user with instructions to exhale at a constant flow rate or constant pressure when exhaling the sample breath.

14. The method of claim 9 wherein prompting the user with instructions to exhale comprises prompting the user to exhale the sample breath for at least 6 to 12 seconds.

15. The method of claim 10 further comprising prompting the user to exhale for a third predetermined period of time into the breath sampling port.

16. The method of claim 9 wherein measuring the biological parameter from the sample breath comprises venting a remainder of the sample breath.

17. The method of claim 9 wherein measuring the biological parameter comprises sensing a CO level from the sample breath.

18. The method of claim 9 wherein measuring the biological parameter comprises sensing a level of H₂, CH₄, CO₂, O₂, or C₃H₆O from the sample breath.

19. An apparatus configured to determine sampling compliance by a user, comprising:
a sampling unit having a breath sampling port;
at least one pressure sensor located within the sampling unit and in communication with the breath sampling port;
a processor in communication with the at least one pressure sensor,
wherein the processor is configured to prompt a user with instructions to exhale a sample breath for a predetermined period of time into the breath sampling port, to measure a pressure change relative to an ambient pressure via the pressure sensor upon sensing the sample breath, and to measure a timing of the pressure change imparted by the sample breath upon the pressure sensor,
wherein the processor is further configured to compare the timing of the sample breath against the predetermined period of time and to further compare an intensity of the pressure change against the ambient pressure.

20. The apparatus of claim 19 wherein the processor is further configured to prompt the user with instructions to hold their breath for at least 10 seconds prior to exhaling the sample breath.

21. The apparatus of claim 19 wherein the processor is further configured to prompt a user with instructions to exhale the sample breath for at least 6 to 12 seconds.

22. The apparatus of claim 19 wherein the processor is further configured to prompt the user when the pressure sensor detects a negative pressure change during the predetermined period of time.

23. The apparatus of claim 19 wherein the processor is further configured to prompt the user when the timing of the sample breath falls outside the predetermined period of time.

24. The apparatus of claim 19 wherein the processor is further configured to prompt the user with a corrective suggestion when the timing of the sample breath falls outside the predetermined period of time or when the intensity of the pressure fails to change relative to the ambient pressure or the pressure is negative.

25. A method of determining sampling compliance by a user, comprising:
prompting the user with instructions to exhale a sample breath into a sampling unit for a predetermined period of time;
receiving the sample breath through a breath sampling port defined on the sampling unit;
measuring a pressure change relative to an ambient pressure via at least one pressure sensor located within the sampling unit and in communication with the breath sampling port upon sensing the sample breath;
measuring a timing of the pressure change imparted by the sample breath upon the at least one pressure sensor via a processor in communication with the at least one pressure sensor;
comparing the timing of the sample breath against the predetermined period of time; and
comparing an intensity of the pressure change against the ambient pressure.

26. The method of claim 25 further comprising prompting the user with instructions to hold their breath for at least 10 seconds prior to prompting the user with instructions to exhale the sample breath.

27. The method of claim 25 wherein prompting the user with instructions to exhale comprises prompting the user with instructions to exhale the sample breath for at least 6 to 12 seconds.

28. The method of claim 25 further comprising prompting the user when the pressure sensor detects a negative pressure change during the predetermined period of time.

29. The method of claim 25 further comprising prompting the user when the timing of the sample breath falls outside the predetermined period of time.

30. The method of claim 25 further comprising prompting the user with a corrective suggestion when the timing of the sample breath falls outside the predetermined period of time or when the intensity of the pressure fails to change relative to the ambient pressure or the pressure is negative.

31. An apparatus configured to determine sampling compliance by a user, comprising:
a sampling unit having a breath sampling port;
a first pressure sensor located in communication with a primary flow path within the sampling unit and in fluid communication with the breath sampling port;
a second pressure sensor located in communication with a secondary flow path within the sampling unit and in fluid communication with the breath sampling port and one or more vent openings; and,
a processor in communication with the first and second pressure sensors, wherein the processor is configured to obtain a first pressure measurement from the first pressure sensor and a second pressure measurement from the second pressure sensor and determine a pressure ratio of the second pressure measurement to the first pressure measurement.

32. The apparatus of claim 31 wherein the processor is further configured to prompt the user with instructions to exhale a sample breath for a predetermined period of time into the breath sampling port.

33. The apparatus of claim 32 wherein the processor is further configured to measure a pressure change relative to an ambient pressure upon sensing the sample breath.

34. The apparatus of claim 33 wherein the processor is further configured to measure a timing of the pressure change imparted by the sample breath.

35. The apparatus of claim 34 wherein the processor is further configured to compare the timing of the sample breath against the predetermined period of time and to further compare an intensity of the pressure change against the ambient pressure.

36. The apparatus of claim 31 wherein the processor is further configured to compare subsequent pressure ratios obtained from subsequent breath sample measurements against the pressure ratio such that deviations of the subsequent pressure ratios relative to the pressure ratio are indicative of the one or more vent openings being obstructed.

37. The apparatus of claim 36 wherein the processor is further configured to prompt the user with a corrective suggestion when the deviations are detected.

38. A method of determining sampling compliance by a user, comprising:
receiving a sample breath through a sampling port defined on a sampling unit such that a first portion of the sample breath flows into a primary flow path and a second portion of the sample breath flows into a secondary flow path and through one or more vent openings;
obtaining a first pressure measurement via a first pressure sensor in the primary flow path;
obtaining a second pressure measurement via a second pressure sensor in the secondary flow path;
determining a pressure ratio of the second pressure measurement to the first pressure measurement via a processor in communication with the first and second pressure sensors; and
comparing the pressure ratio against subsequent pressure ratios obtained from subsequent breath sample measurements for deviations.

39. The method of claim 38 further comprising prompting the user with instructions to exhale a sample breath for a predetermined period of time into the breath sampling port prior to receiving the sample breath.

40. The method of claim 39 further comprising measuring a pressure change relative to an ambient pressure upon sensing the sample breath.

41. The method of claim 40 further comprising measuring a timing of the pressure change imparted by the sample breath.

42. The method of claim 41 further comprising comparing the timing of the sample breath against the predetermined period of time and to further compare an intensity of the pressure change against the ambient pressure.

43. The method of claim 38 further comprising prompting the user with a corrective suggestion when the deviations are detected.
